(19) United States
(12) Patent Application Publication
(10) Pub. No.: US 2010/0036456 A1
(43) Pub. Date: Feb. 11, 2010

(54) System for electrical stimulation of muscles or nerves

(76) Inventor: Winfried Mayr, Modling (AT)

Correspondence Address: Sunstein Kann Murphy & Timbers LLP, 125 SUMMER STREET, BOSTON, MA, 02110-1618, US

(21) Appl. No.: 12/532,306

(22) PCT Filed: Mar. 20, 2008
(86) PCT No.: PCT/AT08/00100

§ 371 Date: Sep. 21, 2009

(30) Foreign Application Priority Data
Mar. 21, 2007 (AT) A 447 2007

(51) Int. Cl. A61N 1/372

(52) U.S. Cl. 607/48

(57) ABSTRACT

The invention relates to a system (1) for the electrical stimulation of muscles, in particular denervated muscles (M), or nerves, comprising a stimulator (2) for generating electrical pulses which is connected to a device (4) for supplying electrical power and for control, and which system (1) also comprises at least two electrodes (3) connected to the stimulator (2) for delivering electrical pulses to the muscles (M) or nerves. In order to create such a stimulation system (1) which results in an improvement with regard to reliability, selectivity and ease of use, the stimulator (2) and at least two electrodes (3) are designed such that they can be implanted, wherein at least one electrode (3) is designed to be fastened to a bone (K). Functional electrical stimulation, in particular for decubitus prophylaxis, can be used in this manner.

The invention relates to a system for the electrical stimulation of muscles, in particular denervated muscles, or nerves, comprising a stimulator for generating electrical pulses which is connected to a device for supplying electrical power and for control, and which system also comprises at least two electrodes connected to the stimulator for delivering electrical pulses to the muscles or nerves.

Triggering muscle contractions by electrical stimulation pulses has been known for a very long time. In this case, a distinction is drawn between both non-invasive appliances, where the electrical pulses are delivered via the skin, and implants with implanted electrodes. Areas of use range from cardiac pacemakers to appliances for reactivating paralyzed muscles of the upper and lower extremities, via cochlear implants and devices for stimulating the bladder and the pelvic floor. In functional electrical stimulation, it is usual for the nerves to be stimulated by electrical pulses at a particular frequency and amplitude, and the muscles connected to the nerves contract as a result of this. Within the scope of more recent research projects it has been established that, contrary to the previous professional opinion, functional electrical stimulation makes possible both efficient build-up and also preservation of muscles in the case of denervated muscles, that is to say after the nerve connections have been severed (W. Mayr et al.: Functional Electrical Stimulation (FES) of Denervated Muscles: Existing and Prospective Technological Solutions, Basic Appl. Myol., 12, 6, 2002: pp 287-290). Functional electrical stimulation of paralyzed patients is an extraordinarily effective prophylaxis against decubitus ulcers which not only put extraordinary amounts of strain on the social and occupational life of the people affected, but can also lead to life-threatening infections. Furthermore, the treatments of such complications cause significant costs and thus strain the health care system.

By way of example, it is possible to electrically stimulate muscle tissue directly by means of surface electrodes arranged on the skin. In this case, pulses of longer pulse duration and with a higher pulse charge, and hence greater continuous electrical power, are applied compared to conventional nerve stimulation. In addition to the electrical dangers which occur due to this and the very limited selectivity of muscle activation, application by means of surface electrodes is cumbersome and in the long run is consistently carried out only by particularly motivated patients. However, in order to efficiently build-up and preserve the muscles, consistent, if possible daily, stimulation of the muscles is necessary.

Conventional concepts for designing systems which can be implanted for the electrical stimulation of denervated muscles are unrewarding for a number of reasons. Since the implanted electrodes need to have a required minimum size to achieve appropriate stimulation they would constitute a foreign body in or on the muscle; this causes strain and would lead to massive connective tissue reactions. Furthermore, there would be further irritation of the soft tissue due to the relative movement of the electrodes with respect to the muscle, and the electrodes would furthermore be exposed to high mechanical loads with a medium-term risk of breakage. The required lines between the electrodes and the stimulator would also be continuously exposed to mechanical loads, in particular tension and bend cycles, so that a very limited life could be expected for the electrodes or the implanted components.

U.S. Pat. No. 5,038,781 A discloses an apparatus for the electrical stimulation of nerves using so-called cuff electrodes, which are electrodes which can be implanted and which surround the nerve. The described system cannot be applied to the direct stimulation of muscle tissue.

U.S. Pat. No. 5,167,229 A discloses a similar system for nerve stimulation or neuromuscular stimulation.

U.S. Pat. No. 5,285,781 A discloses a system for non-invasive neuromuscular stimulation with the aid of surface electrodes.

A method and a system for achieving movements of the extremities by functional electrical stimulation is also disclosed in WO 97/04833 A1, in which surface electrodes are likewise used and pure movement control is intended to be achieved.

A system which can be implanted for the functional electrical stimulation of the nerves is disclosed in US 2003/0139782 A1. Direct stimulation of muscle tissue is not discussed therein.

It is therefore the object of the present invention to create an above-mentioned system for the electrical stimulation of muscles, in particular denervated muscles, or nerves which has an improved design with regard to reliability, selectivity and ease of use compared to conventional systems. It is intended that the disadvantages of the conventional methods are avoided, or at least reduced.

The object according to the invention is achieved by means of an above-mentioned stimulation system, in which the stimulator and at least two electrodes are designed such that they can be implanted, wherein at least one electrode is designed to be fastened to a bone. As a matter of principle, the stimulation system requires at least two electrodes, wherein one electrode can be formed by the stimulator or can be arranged on it. According to the invention, the stimulation system is thus designed such that it can be implanted and at least one electrode is arranged in the vicinity of the muscles on the bony skeleton. Advantageously, all stimulation electrodes are designed to be fastened to a bone. However, applications are also possible in which only one electrode is arranged on a bone and another electrode is arranged in the tissue. By way of example, the electrodes are fastened to the femur for activating the quadriceps and/or the hamstring muscles, or they are fastened to the pelvis for training the gluteus muscles. Since the electrodes are not fastened to or in the muscle to be stimulated, the connective tissue reactions that occur due to this can be reduced, and the mechanical load on the electrode and the connecting lines can be avoided. This results in an increased life of the stimulation system. Furthermore, selective stimulation of the muscle is possible by an appropriate arrangement of the electrodes on the bone underneath. In contrast to stimulation by surface electrodes, substantially lower pulse amplitudes and possibly also lower pulse widths and hence a lower electrical continuous power are required, as a result of which the stimulation system can be operated by batteries even for a relatively long period of time. The implanted stimulation system according to the present description allows more regular use, as a result of which a more efficient build-up and preservation of the degenerated muscle becomes possible, so that the occurrences of decubitus ulcers can be reduced or avoided. The described system is distinguished by particularly robust technical components and protection of the soft tissue. Hence, this provides a cost-effective and long-term stable solution for effective decubitus prophylaxis, as a result of which the health and quality of life of the persons affected are improved, and costs to the health care system can be reduced. In addition, the system according to the subject matter offers advantages with regard to the reliability and acceptance by the patient affected.

As already mentioned above, at least one electrode can be arranged on the stimulator or can be formed by the housing of the stimulator. Designs in which the stimulator comprises two or more electrodes are likewise possible and in which case the stimulation system comprises one module.

According to one feature of the invention, at least one electrode is formed by a substantially plate-like element of electrically conductive material. The size of the plate-like electrode is matched to the respective applications depending on the electrical power intended to be transmitted.

In order to permit optimum fastening to the bone, the shape of at least one electrode is preferably matched to the bone to which it is intended to be fastened.

According to another feature of the invention, at least one of the electrodes intended to be fastened to the bone (K) is designed to be annular, wherein the annulus formed can be arranged around the bone onto which it is intended to be fastened. By way of example, such an annular electrode can be arranged around a tubular bone, such as the femur.

In particular in the case of the last-mentioned annular electrodes it is advantageous if the electrode is of multipart design. This eases arranging the electrode on the respective bone onto which it is intended to be fastened.

It is also possible to match the shape of the electrode to the bone by ensuring that at least one electrode can be deformed. Deformation can be achieved by an appropriate choice of the thickness and an appropriate choice of the material of the electrode.

The electrodes can be formed of metal, in particular stainless steel, titanium or platinum-iridium, or else of an electrically conductive plastic.

Advantageously, each electrode designed to be fastened to a bone is electrically insulated from the bone or the fastening apparatuses fastening it to the bone. On the one hand, this insulation avoids electrical corrosion of components of the stimulation system and tissue damage, and, on the other hand, painful irritation of the periosteum (bone skin) is prevented during the stimulation.

By way of example, the electrical insulation of each electrode designed to be fastened to a bone can be formed by means of an insulation layer.

In order to be able to fasten the electrodes to a bone, apparatuses for fastening it to the bone are advantageously provided.

In order to prevent irritation of the periosteum via the fastening apparatuses, it is also preferable for these fastening apparatuses to be designed to be electrically insulated from the electrode. By way of example, this electrical insulation can be formed by appropriate insulating rings arranged between fastening screws and the electrode.

The fastening apparatuses of the electrodes can be formed in a simple manner by holes for holding bone screws. In order to obtain a planar surface in the implanted state, the holes may have appropriate countersinks for the heads on the bone screws. Instead of holes, it is of course also possible that only recesses are provided in the electrode, in which the bone screws engage in an appropriate manner.

As an alternative to fastening by means of bone screws, the fastening apparatuses can be formed by loops or the like for suturing. The surgeon can thus fasten the electrodes on the periosteum by fitting appropriate sutures.

Furthermore, the fastening apparatuses of the electrodes can be formed by barbs or the like. Such barbs can be provided on the edges of the electrode, or else on elements such as pins connected to the electrode and allow the electrode to be attached to the bone without additional connective means such as screws or the like. The barbed structures can be designed in a number of ways.

At least one electrode can also be designed to be fastened by means of an adhesive on the side which faces the bone as an alternative to fastening it by means of bone screws, sutures, or barbs. Accordingly, the surface of the electrode facing the bone can for example be designed to be rough to form a hold for the adhesive as a connective layer between the bone and the electrode.

The stimulator can also be designed to be fastened to a bone, in a similar manner to the electrodes, by a corresponding design or shape of the surface of the stimulator.

As is the case for the electrodes, it is also possible to provide apparatuses on the stimulator for fastening it to the bone.

By way of example, it is also possible for these fastening apparatuses of the stimulator to be formed by holes for holding bone screws.

The fastening apparatuses of the stimulator can also be formed by barbs or the like.

Additionally or as an alternative, it is possible for the stimulator to be designed to be fastened by means of an adhesive on the side which faces the bone. By way of example, the surface of the stimulator facing the bone therefore can be roughened so that a firm connection between the bone and the stimulator can be created by means of an adhesive, as is the case for the electrode.

According to a further feature of the invention, the supply and control device of the stimulator system is also designed such that it can be implanted. Thus, a stimulation system is created which can be completely implanted and which can work autonomously.

According to a further feature of the invention, the supply and control device designed such that it can be implanted comprises a rechargeable battery. By way of example, this rechargeable battery which can be implanted can be recharged without the use of wires overnight so that on the next day there is once again sufficient energy available for stimulation.

As an alternative, it is possible to provide a receiving coil connected to the stimulator, and the supply and control device can be connected externally to an emitting coil for supplying electrical power to the stimulator, and for controlling the stimulator, without the use of wires. This design of the stimulation system provides for the relatively small components which can be fitted, the stimulator, electrodes and receiving coil, to be implanted in the patient, whereas the supply and control device, usually relatively large, and the emitting coil are arranged externally. The electrical power is supplied to the stimulator inductively by means of the emitting coil and receiving coil of the system. As a result of this, components which penetrate the patient's skin are not required.

In the above-mentioned embodiment, fastening elements are advantageously provided for fastening the receiving coil of the implanted part of the stimulation system to the bone. These fastening elements can be clamps with appropriate holes for holding bone screws, with the aid of which the receiving coil can be fastened to the bone under the muscle tissue to be stimulated.

In order to avoid irritation of the muscle tissue, the stimulator and the receiving coil can be designed to be arranged in a cut-out in the bone. In this case, the surgeon would use appropriate tools to mill into the bone a cut-out corresponding to the size of the stimulator and, if appropriate, the receiving coil and the electrodes, and subsequently arrange the stimulator and, if appropriate, the receiving coil and the electrodes in this cut-out of the bone and fasten it or them by means of adhesives or screws, for example.

So as to permit frequent application which does not put a lot of strain on the patient, the emitting coil of the stimulation system can be integrated in the backrest and/or the seat of a wheelchair. In this manner, the paralyzed patient equipped with the corresponding implanted components of the stimulation system can be stimulated at appropriate time intervals while sitting in the wheelchair, and, in this manner, a build-up and preservation of the denervated muscles can be achieved.

Emitting coils can likewise also be integrated in a bed, as a result of which the patient equipped with the implanted components of the stimulation system can also be stimulated while sleeping, for example. As a result, the acceptance is further increased and more frequent application of the functional electrical stimulation and, as a result, improved build-up and preservation of the muscles are made possible.

Finally, it is also possible for the emitting coil of the stimulation system to be integrated in a piece of clothing. By way of example, it is possible that the belt, a pair of trousers, a jacket or the like could be suitable for this purpose, with the at least one emitting coil being arranged according to the implanted components of the stimulation system.

The invention also relates to a method for the electrical stimulation of denervated muscles using a system described above.

The subject matter of the invention will be described in more detail on the basis of the appended drawings, which show outline sketches and embodiments of the invention, and in which

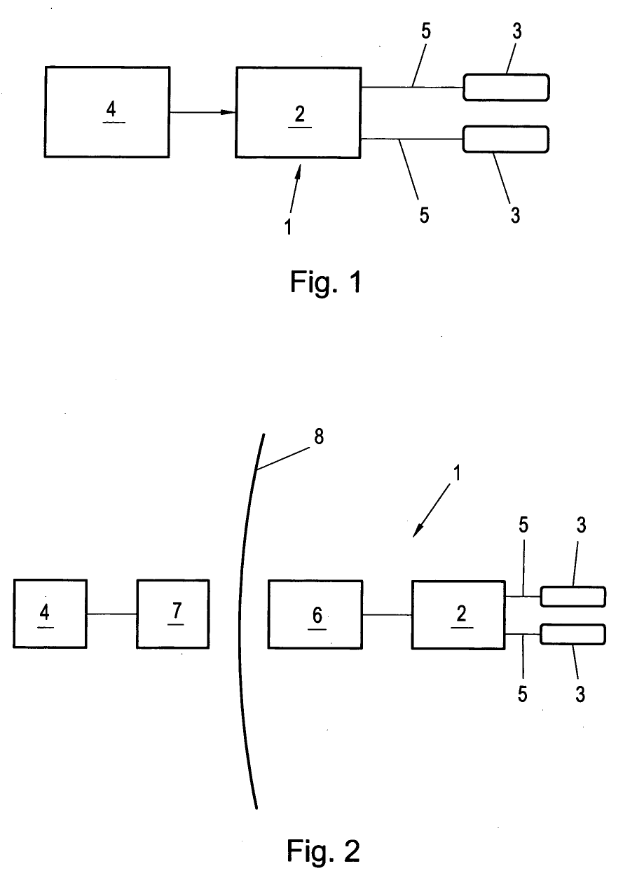

FIG. 1 shows an outline block diagram of the most important components of a system for functional electrical stimulation;

FIG. 2 shows an outline block diagram of a system for functional electrical stimulation with electrical power transferred without the use of wires;

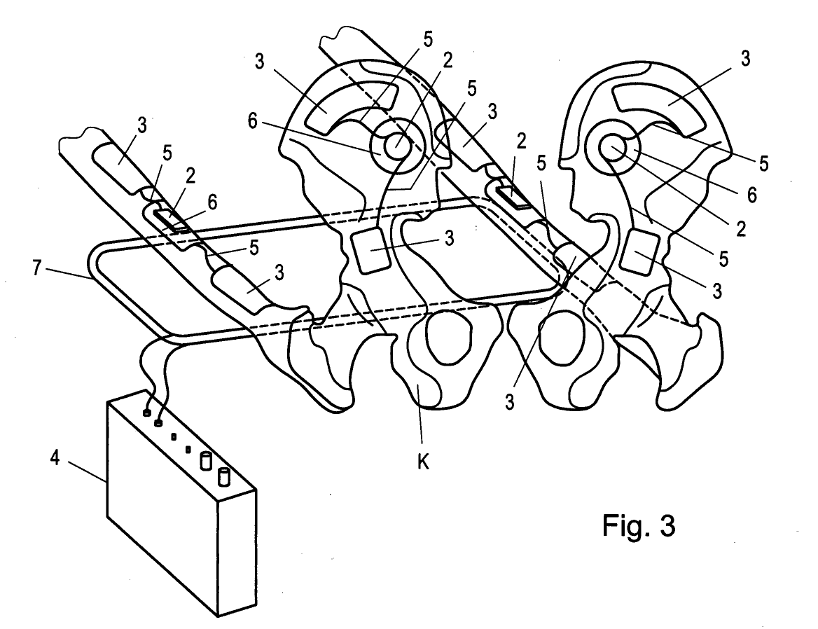

FIG. 3 shows an outline sketch of the possible arrangement of the electrodes and the stimulator on the femur and the pelvic bone of a patient;

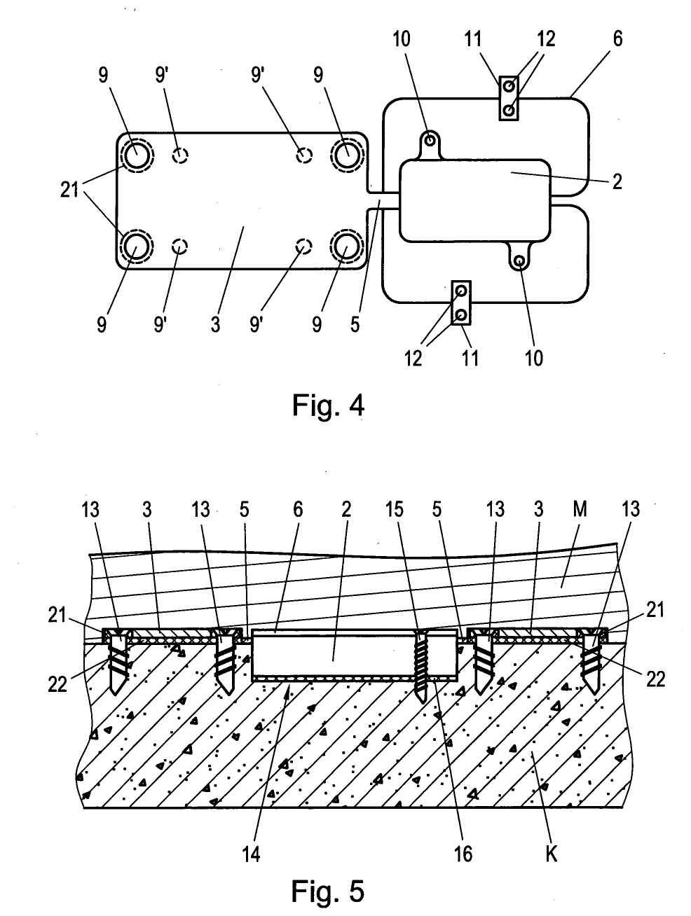

FIG. 4 shows an embodiment of the components of the stimulation system which can be implanted;

FIG. 5 shows a section through a further embodiment of the stimulation system at a location fastened on the bone;

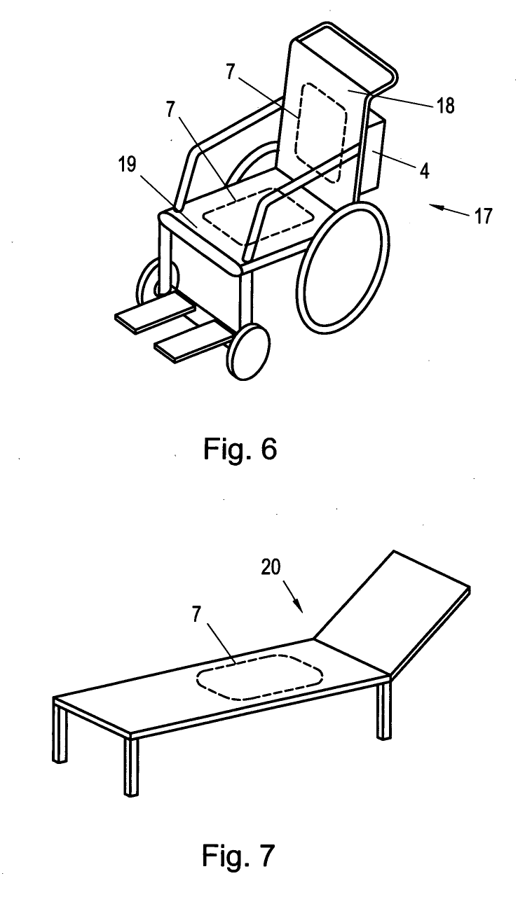

FIG. 6 shows the arrangement of emitting coils of the stimulation system in a wheelchair; and

FIG. 7 shows the arrangement of an emitting coil of the stimulation system in a bed.

FIG. 1 shows an outline block diagram of a system 1 for the electrical stimulation of muscles, in particular denervated muscles, or nerves, comprising a stimulator 2 for generating electrical pulses which is connected to a device 4 for supplying electrical power and for control. The stimulator 2 is connected via appropriate connecting lines 5 to at least two electrodes 3 which deliver the electrical pulses to the respective tissue. In place of the connecting line 5, it is also possible for the electrodes 3 to be designed such that they can be connected directly to the stimulator 2. Furthermore, at least one electrode 3 can be arranged on the stimulator 2, or can be formed by the housing of the stimulator 2 (not shown). It is likewise possible for the stimulator 2 to comprise at least two electrodes 3, and the stimulator 2, together with the at least two electrodes 3, thus forms a closed unit (not illustrated).

FIG. 2 shows a further outline block diagram of a stimulation system 1, in which the connection between the stimulator 2 and the supply and control device 4 does not use wires. Correspondingly, the stimulator 2 is connected to a receiving coil 6 and the supply and control device 4 is connected to an emitting coil 7 by means of which the appropriate electrical power is transferred inductively to the receiving coil 6. According to the present invention, the stimulator 2, the at least one electrode 3 together with the connecting line 5, and the receiving coil 6 are designed such that they can be implanted, whereas the supply and control device 4 and the emitting coil 7 are arranged outside the body. The line 8 correspondingly denotes the skin surface of the body in FIG. 2.

FIG. 3 shows an outline arrangement of the stimulators 2 and electrodes 3 which can be implanted on the bony skeleton of a patient, in which respective modules comprising a stimulator 2 and two electrodes 3, and also a receiving coil 6, are fastened to the femur or pelvic bone K and permit optimum direct stimulation of the muscles M lying above the bone K (see FIG. 5). The supply and control device 4 is connected to the emitting coil 7 and supplies electrical power to the receiving coil 6 or receiving coils 6 as required for the stimulation without the use of wires. Once the appropriate components of the stimulation system 1 have been implanted in the patient, the electrical stimulation can be carried out relatively simply, and it can be carried out without relevant additional effort for the patient. In particular, the emitting coil 7 can for example be integrated in the seat pad or backrest of a wheelchair (see FIG. 6), which is why the patient need only start the stimulation program at regular intervals. In the case of regular application of the functional electrical stimulation, a quick build-up of the degenerated muscle and its preservation is possible, and the formation of decubitus ulcers can be reduced or effectively prevented. For this purpose, the training units can also be fully-automatically time-controlled.

FIG. 4 shows an embodiment of the components of the stimulation system 1 which can be implanted, comprising the stimulator 2, an electrode 3 and a receiving coil 6. By way of example, the opposing electrode can be arranged on the stimulator 2 (not illustrated). The electrode 3 has fastening apparatuses for fastening it on the bone and which, for example, can be formed by holes 9 for holding bone screws 13 (see FIG. 5) or by loops 9′ or the like for suturing. The holes 9, loops 9′ or the like are also suitable for the growing in of connective tissue, as a result of which the electrode 3 can be fastened to the bone. In the illustrated example, the connecting line 5 between the electrode 3 and stimulator 2 is formed by a correspondingly elongate design of the electrode 3, as a result of which weak points such as soldered or terminal connections of wires on the electrode 3 can be avoided. In order to prevent electrochemical corrosion on the bone screws 13 via the fastening apparatuses or irritation of the periosteum, the holes 9 for holding bone screws 13 can be electrically insulated from the electrode 3. By way of example, this electrical insulation can be formed by corresponding insulating rings 21.

The stimulator 2 can also be provided with apparatuses for fastening it on the bone and which can in turn be formed by corresponding holes 10 for holding bone screws 15 (see FIG. 5). It is likewise possible to provide fastening elements 11 to fasten the receiving coil 6 on the bone and which in turn can have holes 12 for holding bone screws (not illustrated).

FIG. 5 shows a further embodiment of the implanted part of a stimulation system according to the present application, in which the stimulator 2 is at least partially arranged in a cut-out 14 in the bone K. The electrodes 3 can likewise be arranged in corresponding cut-outs in the bone (not illustrated), so that a planar surface is created on the bone K and there is no source of irritation for the muscle M. The electrodes 3 and the stimulator 2 can be fastened to the bone K by means of appropriate bone screws 13, 15 or with the aid of an adhesive 16. In the illustrated example, the receiving coil 6 is arranged on the upper side of the stimulator 2 facing away from the bone K and is integrated in the stimulator 2. Rather than using bone screws 13, 15, it is also possible to arrange pin-like structures with barbs on the electrodes 3 or the housing of the stimulator 2, and to fasten it or them to the bone K by these means. Barbs can also be arranged on the edges of the electrodes 3 themselves or the housing of the stimulator 2 itself. To avoid electrical corrosion and tissue damage, and painful irritation of the periosteum, each electrode 3 designed to be fastened to the bone K is preferably electrically insulated from the bone K. By way of example, this electrical insulation can be formed by an insulation layer 22 of electrically insulating material.

FIG. 6 shows an outline sketch of a suitable arrangement of the emitting coils 7 of the stimulation system in the backrest 18 and/or the seat 19 of a wheelchair 17. The emitting coils 7 are appropriately connected to the supply and control device 4. In this manner, it is possible to stimulate the muscles of the patient at those locations at which decubitus ulcers occur most frequently, while the patient is seated in the wheelchair 17.

FIG. 7 illustrates that it is also possible to place or integrate an emitting coil 7 of the stimulation system 1 in a bed 20.

Furthermore, the emitting coil 7 of the stimulation system 1 can also be arranged in a piece of clothing, so that comfortable and regular application is made possible.

1.-30. (canceled)

31. A system for the electrical stimulation of muscles, in particular denervated muscles, or nerves, the system comprising:
an implantable stimulator for generating electrical stimulation pulses;
a supply and control device for supplying electrical power and for control of the system;
at least two implantable electrodes connected to the stimulator for delivering the electrical pulses to adjacent muscle tissue
wherein at least one electrode is adapted to match the shape of an anchoring bone to which the at least one electrode is attached.

32. A stimulation system as in claim 31, wherein the stimulator has an outer surface including at least one of the electrodes.

33. A stimulation system as in claim 31, wherein the at least one electrode is a substantially plate-like element of electrically conductive material.

34. A stimulation system as in claim 31, wherein the at least one of the electrode has an annular shape adapted to be arranged around the anchoring bone.

35. A stimulation system as in claim 31, wherein the at least one electrode is deformable to match the shape of the bone.

36. A stimulation system as in claim 31, wherein the implantable electrodes are formed of an electrically conductive plastic.

37. A stimulation system as in claim 31, wherein the at least one electrode is electrically insulated from the anchoring bone.

38. A stimulation system as in claim 31, wherein an adhesive is used for fastening the at least one electrode to the bone.

39. A stimulation system as in claim 31, wherein the stimulator also is adapted to be fastened to a stimulator anchoring bone.

40. A stimulation system as in claim 39, wherein an adhesive is used for fastening the stimulator to the stimulator anchoring bone.

41. A stimulation system as in claim 31, wherein the supply and control device is implantable.
